(19) United States
(12) Patent Application Publication
(10) Pub. No.: US 2005/0159633 A1
(43) Pub. Date: Jul. 21, 2005

(54) Use of alkali metal doped eta-alumina as methanol hydrochlorination catalyst

(75) Inventors: Christopher J. Mitchell, Veltem-Beisem (BE); Peter Jones, Warrington (GB)

Correspondence Address: MAYER, BROWN, ROWE & MAW LLP, 1909 K STREET, N.W., WASHINGTON, DC, 20006, US

(73) Assignee: Ineos Chlor Limited, Runcorn (GB)

(21) Appl. No.: 11/017,008

(22) Filed: Dec. 21, 2004

Related U.S. Application Data
(63) Continuation of application No. 09/959,399, filed on Jun. 10, 2002, now abandoned.

(30) Foreign Application Priority Data
Jun. 15, 1999 (GB) 9913758.0
Nov. 3, 1999 (GB) 9925940.0

(51) Int. Cl. C07C 17/16

(52) U.S. Cl. 570/258

(57) ABSTRACT

A catalyst for use in the hydrochlorination of methanol which comprises an η-alumina doped with an alkali metal salt, e.g. caesium chloride, in order to reduce selectivity to demethyl ether and to delay the onset of coking on the catalyst in use.

The present invention relates to catalysts for use in the preparation of methyl chloride and to a process for the preparation of methyl chloride from methanol and HCl using such catalysts. The invention is also concerned with a process for extending the active life of such catalysts.

In the commercial production of methyl chloride using a gas phase catalytic process, methanol and hydrogen chloride are typically fed in an approximately equimolar ratio to a fixed bed or fluidised bed reactor at a temperature of 250-300° C. The reaction is exothermic and large temperature rises are often observed, with temperatures of over 400° C. being readily obtained. Such high temperatures, or hot spots, can lead to catalyst sintering and coke formation, with consequent loss in catalyst activity, over relatively short periods of time. The operating pressure of commercial reactors is not critical to the operation of the process: low and high pressure reactors are used.

Alumina is commonly used as the catalyst for the production of methyl chloride from methanol and HCl. Usually, γ-alumina is the preferred catalyst as acceptable levels of activity for methyl chloride formation are obtained, without the generation of excessive hot spots within the catalyst bed. For example, U.S. Pat. No. 5,183,797 teaches the use of γ-alumina catalysts for the production of methyl chloride with a controlled reaction hot spot to limit catalyst coking by controlling the surface area of the catalyst.

The major by-product from the reaction of methanol with hydrogen chloride is dimethyl ether.

We have now found that where η-alumina doped with an alkali metal salt is used as catalyst in the reaction of methanol with hydrogen chloride a significant reduction in selectivity to dimethyl ether is obtained. The selectivity to dimethyl ether tends to be approximately 100 times lower than that obtained with a commercially available γ-alumina catalyst.

According to a first aspect of the present invention there is provided a catalyst for the hydrochlorination of methanol which comprises an η-alumina doped with an alkali metal salt.

According to a second aspect of the present invention there is provided a catalyst for the hydrochlorination of methanol, the preparation of which includes the step of doping an η-alumina with an alkali metal salt. Thereafter the doped product may be calcined.

According to a third aspect of the present invention there is provided a process for the preparation of methyl chloride which comprises treating methanol with HCl in the vapour phase in the presence of a catalyst as defined in the first or second aspects of the present invention.

Preferably the alkali metal in the alkali metal salt with which the η-alumina is doped according to the present invention is caesium or potassium, more preferably caesium, since the reduction in selectivity to dimethyl ether is more marked. We have found that there is little difference in the selectivity to methyl chloride or dimethyl ether between different salts of the same alkali metal, for example nitrate, chloride and hydroxide.

Doping of the η-alumina with the alkali metal salt may be effected by impregnation techniques known in the art. Typically an aqueous solution of the alkali metal salt is added dropwise to the η-alumina. The η-alumina is then heated under vacuum to remove the water. The doped catalyst may then be calcined.

According to a further aspect of the present invention there is provided a process for the preparation of a catalyst according to the first or second aspects of the present invention which process comprises the step of impregnating an η-alumina with an aqueous solution of an alkali metal salt.

The concentration of the aqueous alkali metal salt solution used in the process according to the further aspect of the present invention will be chosen to give the desired concentration of alkali metal salt in the catalyst.

The concentration of alkali metal salt in the catalyst is typically 0.05-5.0 mmolg⁻¹ preferably 0. 1-3.0 mmolg⁻¹, and more preferably 0.1-2.0 mmolg⁻¹, e.g. 0.2-2.0 mmolg⁻¹.

The physical form of the catalysts, eg shape and size, is chosen in the light of inter alia the particular reactor used in the hydrochlorination reaction and the reaction conditions used therein.

The molar ratio of HCl: methanol used in the preparation of methyl chloride is at least 1:10 and no greater than 10:1 preferably 1:1.5-1.5:1, more preferably approximately stoichiometric.

In such preparation, the process may be carried out at 200-450° C., preferably about 250° C.

In such preparation, the process may be carried out in high pressure or low pressure vapour phase hydrochlorination reactors, typically at between 1 and 10 bara.

The preparation process may be carried out batch-wise or as a continuous process. A continuous process is preferred.

Further aspects of the invention are concerned with the doping of η-alumina in order to delay the onset of coking on such a catalyst when used in a hydrochlorination reaction.

Preferably in such further aspects of the invention, the alkali metal in the alkali metal salt with which the η-alumina is doped is caesium or potassium, more preferably caesium. We have found that there is little difference in the rate of coke formation between different salts of the same alkali metal, for example nitrate, chloride and hydroxide.

The present invention is further illustrated by reference to the following Examples.

The performance of the catalysts in the hydrochlorination of methanol was evaluated using a conventional microreactor system operating at atmospheric pressure, with gas flows controlled via Brooks mass flow controllers.

In the Examples, surface areas and pore volumes of the catalysts were measured by nitrogen absorption and catalyst activities were measured in a laboratory microreactor. The nitrogen absorption isotherms were measured using a Micromeritics ASAP2400 Gas Absorption Analyser, after out-gassing of the catalyst samples overnight.

General Procedure

Approximately 0.04 ml/min liquid methanol was fed via a HPLC pump to a stainless steel vaporiser packed with 2-3mm diameter glass beads held at a temperature of 130° C. The vaporised methanol flow obtained in this manner was equivalent to 36.3 ml/min methanol vapour flow at room temperature and pressure. To assist with the flow of methanol through the vaporiser, 25 ml/min nitrogen gas was co-fed to the vaporiser. The vaporised methanol/nitrogen mixture was mixed with 40ml/min hydrogen chloride gas, and fed to a U-shaped pyrex reactor tube containing catalyst and held within an air-circulating oven. The temperature of the oven was monitored via two thermocouples placed against the reactor wall in the vicinity of the packed catalyst bed.

In Examples 1 to 14, the catalyst extrudates were crushed and sieved to a 300 to 500 micron size fraction and of the crushed catalyst 0.07g was mixed with 0.9g pyrex of a similar size fraction. This mixture was placed in the reactor tube of the microreactor system within the oven at a temperature of 250° C. Catalyst performance was evaluated by increasing the temperature of the oven at 10° C./hr up to a maximum temperature of 310° C. Samples of the reactor products were analysed by gas chromatography every 15 minutes.

The exit gases from the microreactor were mixed with 5 L/min nitrogen gas to prevent any reaction products or unreacted methanol from condensing, and a portion of this stream was analysed by gas chromatography using a HP5890 Gas Chromatograph fitted with a gas-sampling valve and 50 m×0.530 mm diameter CPWax 52 capillary column (ex Chrompak). The signal obtained from the gas chromatograph was integrated using PE Nelson Turbochrom software, and the relative composition of methyl chloride, dimethyl ether and unreacted methanol were reported as a normalised %v/v composition using relative response factors for these components, which had been previously determined from analysis of volumetrically prepared standard gas mixtures.

The results of the temperature profiles were analysed using a linearised form of the Arrhenius equation (a ln(%v/v) versus 1/T plot), to give estimated values for the activity for methyl chloride and dimethyl ether formation at 290° C.

### EXAMPLES 1-3

These Examples are Comparative Tests using γ-alumina extrudates with surface areas of 296 m²g⁻¹, 196 m²g⁻¹ and 225 m²g⁻¹ respectively crushed and sieved to a 300-500 micron size ftaction. Evaluation of their performance yielded the results shown in Table 1.

TABLE 1

| | | | Dimethyl | Methyl |
|---|---|---|---|---|
| Example | | Surface Area | Ether | Chloride |
| No. | Catalyst | (m²g⁻¹) | (% v/v) | (% v/v) |
| 1 | γ-alumina | 296 | 2.30 | 21.6 |
| 2 | γ-alumina | 195 | 3.10 | 24.10 |
| 3 | γ-alumina | 225 | 2.30 | 18.50 |

From Table 1 it can be seen that (a) these catalysts show acceptable levels of activity towards methyl chloride formation, with a significant level of by-product dimethyl ether formation and (b) the activities of these catalysts are not directly related to the measured surface areas.

### EXAMPLES 4-6

These Examples are Comparative Tests in which η-alumina extrudates with BET surface areas of 332 m²g⁻¹, 417 m²g⁻¹ and 398 m²g⁻¹ respectively were crushed and sieved to a 300-500 micron size fraction and their performance evaluated. The results obtained are shown in Table 2.

TABLE 2

| | | Surface | Dimethyl | Methyl |
|---|---|---|---|---|
| Example | | Area | Ether | Chloride |
| No. | Catalyst | (m²g⁻¹) | (% v/v) | (% v/v) |
| 4 | η-alumina | 332 | 2.70 | 53.9 |
| 5 | η-alumina | 417 | 2.90 | 50.60 |
| 6 | η-alumina | 398 | 2.3 | 54.30 |

From Table 2 it can be seen that the levels of activity for methyl chloride formation obtained with the η-alumina catalysts are significantly higher than those obtained with the γ-alumina catalysts (Examples 1-3), whilst the levels of dimethyl ether obtained are similar to those observed with the γ-alumina catalysts. It will be appreciated that with these high levels of activity towards methyl chloride formation, the use of such η-alumina catalysts in an industrial process becomes problematic owing to the generation of large hot spots within the catalyst bed.

### EXAMPLES 7 and 8

These Examples illustrate the use of doped η-aluminas according to the present invention. Samples of the η-alumina extrudates used in Example 4 were impregnated with potassium chloride and caesium chloride in the following manner. η-alumina extrudate (approximately 10 g) was added to a two necked flask, and the flask was evacuated to remove the air from the pores of the alumina. Alkali metal salt solution (approximately 30 ml³ was added to the flask via a dropping funnel. The catalyst particles were then filtered off and dried on a rotary evaporator at 70° C. under vacuum for one hour. After drying, the catalysts were crushed and sieved to a 300-500 micron particle size fraction for evaluation. The nominal alkali metal loading of each catalyst sample was calculated from the measured pore volume of the η-alumina extrudate, and the concentration of the salt solution used for each preparation. The results obtained are shown in Table 3.

TABLE 3

| | | Surface | Dimethyl | Methyl |
|---|---|---|---|---|
| | | Area | Ether | Chloride |
| Example | Catalyst | (m²g⁻¹) | (% v/v) | (% v/v) |
| 7 | η-alumina + 1.0 mmolg⁻¹ | 241 | 0.12 | 16.20 |
| | Kcl | | | |
| 8 | η-alumina + 1.0 mmolg⁻¹ | 166 | 0.03 | 14.20 |
| | CsCl | | | |

From Table 3 it can be seen that (a) the addition of the alkali metal salt has moderated the activity for methyl chloride formation to acceptable levels whilst the selectivity to dimethyl ether has been dramatically reduced and (b) the effect of the caesium salt on selectivity to dimethyl ether is significantly greater than that obtained with the potassium salt.

### EXAMPLES 9 and 10

These Examples illustrate catalysts according to the present invention comprising η-alumina doped with caesium chloride. Samples of the η-alumina catalysts used in Examples 5 and 6 were impregnated with caesium chloride in the manner described in. Examples 7 and 8. The results obtained are shown in Table 4.

TABLE 4

| | | Surface | Dimethyl | Methyl |
|---|---|---|---|---|
| Example | | Area | Ether | Chloride |
| No. | Catalyst | (m²g⁻¹) | (% v/v) | (% v/v) |
| 9 | η-alumina + 1.0 mmolg−1 | 93 | 0.02 | 22.0 |
| | CsCl | | | |
| 10 | η-alumina + 1.0 mmolg−1 | 83 | 0.02 | 24.70 |
| | CsCl | | | |

From Table 4 it can be seen that addition of the caesium salt has moderated the activity towards methyl chloride formation and dramatically reduced the selectivity towards dimethyl ether formation.

### EXAMPLES 11-14

These Examples illustrate further catalysts according to the present invention. In these Examples, samples of the η-alumina extrudate used as Example 4 were impregnated with varying levels of caesium chloride using the method described in Examples 7 and 8. The results obtained shown in Table 5.

TABLE 5

| | | Surface | Dimethyl | Methyl |
|---|---|---|---|---|
| Example | | Area | Ether | Chloride |
| No. | Catalyst | (m²g⁻¹) | (% v/v) | (% v/v) |
| 11 | η-alumina + 0.1 mmolg−1 | 313 | 0.55 | 19.50 |
| | CsCl | | | |
| 12 | η-alumina + 0.3 mmolg−1 | 294 | 0.09 | 14.50 |
| | CsCl | | | |
| 13 | η-alumina + 0.6 mmolg−1 | 232 | 0.03 | 15.40 |
| | CsCl | | | |
| 14 | η-alumina + 1.0 mmolg−1 | 166 | 0.02 | 14.7 |
| | CsCl | | | |

From Table 5 it can be seen that the effect of caesium chloride addition on the observed changes in activity for methyl chloride and dimethyl ether is clearly non-linear. A substantial moderation of the activity for methyl chloride formation is obtained with a 0.1 mmolg^{31 1} caesium chloride loading, but higher levels of caesium chloride are needed to obtain the fullest reduction in selectivity towards dimethyl ether formation.

### EXAMPLES 15-20

These Examples illustrate coking of the catalysts over time, Example 15 being a Comparative Example. In Examples 15-20, catalyst preparation was effected by impregnating η-alumina extrudates with a BET surface area of 320 m²g⁻¹ with caesium chloride in the following manner. η-alumina extrudate (approximately 10 g) was added to a two necked flask, and the flask was evacuated to remove the air from the pores of the alumina. Caesium chloride solution (approximately 30 ml³ ) was added to the flask via a dropping funnel. The catalyst particles were then filtered off and dried on a rotary evaporator at 70° C. under vacuum for one hour. After drying. the catalysts were crushed and sieved to a 300-500 micron particle size fraction for evaluation. The nominal alkali metal loading of each catalyst sample was calculated from the measured pore volume of the η-alumina extrudate, and the concentration of the salt solution used for each preparation.

The coking of the catalysts over time was determined using a Rupprecht and Patashnick PMA1500 Pulse Mass Analyser TEOM reactor system (TEOM refers to a Tapered Element Oscillating Microbalance) A sample (approx. 100 mg) of the catalyst shown in Table 6 was charged to the TEOM reactor and the sample dried in situ under a helium gas flow for 5 hours at 400° C. After drying the temperature of the sample was reduced to 390° C. and kept at this temperature overnight.

TABLE 6

| Example | | Surface Area | Pore |
|---|---|---|---|
| (Curve No.) | Catalyst | (m²g⁻¹) | Volumes (cc/g) |
| 15* (1) | η-alumina | 320 | 0.36 |
| 16 (2) | η-alumina + 0.05 mmol/g | 307 | 0.35 |
| | CsCl | | |
| 17 (3) | η-alumina + 0.10 mmol/g | 307 | 0.34 |
| | CsCl | | |
| 18 (4) | η-alumina + 0.20 mmol/g | 299 | 0.32 |
| | CsCl | | |
| 19 (5) | η-alumina + 0.60 mmol/g | 244 | 0.27 |
| | CsCl | | |
| 20 (6) | η-alumina + 1.00 mmol/g | 181 | 0.21 |
| | CsCl | | |

15* is a Comparative Test

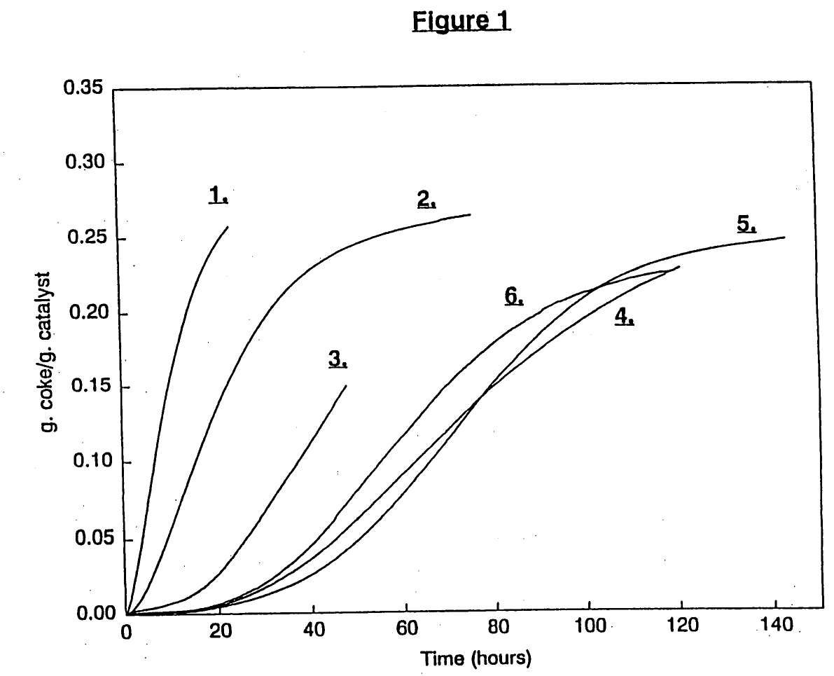

Coking of the catalyst at 390° C. was effected by replacing the He gas flow with methyl chloride (15 ml/min at STP) delivered via a Brooks mass flow controller and monitoring the increase in mass of catalyst over a period of several days at atmospheric pressure. The results are shown in FIG. 1, which shows the mass gain per gram of catalyst as a function of run time. In FIG. 1, the curves depicted by reference numerals 1-6 correspond to Examples 15-20 respectively.

From FIG. 1 it can be seen that for all the catalysts the rate of coke laydown varies in a non-linear manner as a function of time. However, with increasing caesium loading the onset of coking is delayed such that the time to reach a given level of coke is dramatically increased. The data obtained shows that the maximum effect on coke laydown is achieved with a caesium loading greater than or equal to 0.2 mmol/g.

1. A process for the preparation of methyl chloride which comprises treating methanol with HCl in the vapour phase in the presence of a catalyst;
in which the catalyst comprises an η-alumina doped with an alkali metal salt.

2-18. (canceled)
